(19) United States
(12) Patent Application Publication
(10) Pub. No.: US 2023/0127119 A1
(43) Pub. Date: Apr. 27, 2023

(54) SYSTEMS AND METHODS FOR PRODUCING MIXTURES

(71) Applicants: Boston Scientific Medical Device Limited, Galway (IE); Boston Scientific Scimed, Inc., Maple Grove, MN (US)

(72) Inventors: Benjamin CLEVELAND, Bellingham, MA (US); Nitesh Ghananil BAVISKAR, Kalyan West (IN); Junaid Mohammed SHAIKH, Surat (IN); Richard Earl GRAFFAM, Pelham, NH (US); Joseph HERNANDEZ, Rutland, MA (US); Christopher WATSON, Lincoln, MA (US); Jennie CREEGAN, Minneapolis, MN (US); Kolbein KOLSTE, Acton, MA (US); Nicholas DeSANTIS, Cambridge, MA (US); Eric KIM, Brighton, MA (US); Mei Lee AMEND, Lakeville, MA (US); Subodh MOREY, Ponda (IN)

(73) Assignees: Boston Scientific Medical Device Limited, Galway (IE); Boston Scientific Scimed, Inc., Maple Grove, MN (US)

(21) Appl. No.: 18/048,517

(22) Filed: Oct. 21, 2022

Related U.S. Application Data
(60) Provisional application No. 63/270,845, filed on Oct. 22, 2021.

(51) Int. Cl. A61J 1/20

(52) U.S. Cl. CPC A61J 1/2068; A61J 1/2048; A61J 1/2006

(57) ABSTRACT

A system is disclosed for producing a mixture to deliver to a treatment site. The system can include a connector with a connector vent so that air in the connector is expellable therethrough. A multi-lumen chamber can be connected to a proximal end of the connector and include a first lumen aligned and adjacent a second lumen. The first lumen can be configured to include a first constituent in a proximal portion of the first lumen, a second constituent in a distal portion of the first lumen, and a first plunger internally positioned within the first lumen to distally move the first constituent into the distal portion to mix with the second constituent in a first state to form a first mixture. The second lumen is configured to include a third constituent and a second plunger rod internally positioned within the second to distally move the third constituent.

### CROSS-REFERENCE TO RELATED APPLICATIONS

This patent application claims the benefit of priority under 35 U.S.C. § 119 to U.S. Provisional Patent Application No. 63/270,845, filed Oct. 22, 2021, which is herein incorporated by reference in its entirety.

### FIELD

The present disclosure relates generally to compositions for injection to a patient, methods of preparation and use thereof, and devices comprising such compositions.

### BACKGROUND

Numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Since the conception of conformal radiotherapy, physicians have paid attention to the delivered dose to the target and surrounding tissues. Investigators have been able to correlate side effects to the amount of tissue receiving a certain radiation dose. And yet, time, distance, and shielding affect the dose that is delivered. The less time an area is exposed to radiation, the less dose delivered. The greater the distance from the radiation, the less dose delivered.

Current systems provide filler material to treatment sites to decrease the radiation dose to the rectum during radiotherapy for prostate cancer. However, the system that mixes the filler material in vitro includes numerous subcomponents, is complex to assemble, and rife with filler mixing errors prior to delivery within a patient at a treatment site. During the foregoing procedures, such errors and mishaps lead unnecessarily to patient risk, increased procedure time, and increased procedure costs. The solution of this disclosure resolves these and other issues of the art.

### SUMMARY

In accordance with certain embodiments of the present disclosure, a gel composition mixing system is disclosed. The system can include a connector including a central lumen attachable to a proximal end of a delivery system. The connector can include a connector vent in fluid communication with the central lumen configured so that air in the connector is expellable therethrough. A multi-lumen chamber can be connected to a proximal end of the connector and include a first lumen aligned and adjacent a second lumen. The first lumen is configured to include a first constituent in a proximal portion of the first lumen and a second constituent in a distal portion of the first lumen. A first plunger can be internally positioned within the first lumen to distally move the first constituent into the distal portion to mix with the second constituent in a first state to form a first mixture. The first lumen can terminate in a first port. The second lumen can be configured to include a third constituent. A second plunger rod can be internally positioned within the second to distally move the third constituent. The second lumen can terminate in a second port. Distally moving the second plunger rod can cause the third constituent to be delivered through the first and second ports, vent air from system through the connector vent, mix within the central lumen of the connector to form the mixture, and delivered through the delivery system.

In accordance with certain embodiments of the present disclosure, the second plunger rod distally moves the third constituent and the first mixture in the second state.

In accordance with certain embodiments of the present disclosure, distally moving the second plunger rod causes the first mixture and the third constituent to be delivered through the first and second ports while venting air from the system through the connector vent.

In accordance with certain embodiments of the present disclosure, at least one of the first and second lumens includes a floating plunger. At least one of the first and second floating plungers are toggleable when a predetermined pressure is achieved within a respective lumen.

In accordance with certain embodiments of the present disclosure, the delivery system includes a needle assembly removably connected to the connector, the needle assembly including the needle and a needle hub positioned at a proximal end of the needle.

In accordance with certain embodiments of the present disclosure, at least one of the first and second lumens include a floating plunger, wherein the first lumen includes an internally positioned rib at or adjacent a distal end of the first lumen. As the first plunger rod is advanced distally, the first floating plunger advances distally and contacts the rib to develop a moment on a head of the first floating plunger causing the first floating plunger to tilt and break a fluid seal so fluid within the first lumen can advance distal of the first floating plunger.

In accordance with certain embodiments of the present disclosure, the connector vent extends orthogonally from the central lumen to outside air distal of a static mixer.

In accordance with certain embodiments of the present disclosure, the first lumen includes a vent proximal of the first port and orthogonal to the first lumen configured to permit egress of air from the first lumen.

In accordance with certain embodiments of the present disclosure, the first mixture and the third constituent mixed together forms a hydrogel, and wherein the connector vent is configured to prevent air bubbles from entering the hydrogel.

In accordance with certain embodiments of the present disclosure, the connector vent includes an air-permeable-fluid-impermeable membrane.

In accordance with certain embodiments of the present disclosure, the first lumen and the second plunger rod are included in a plunger assembly. The plunger assembly is nestable within the second lumen.

In accordance with certain embodiments of the present disclosure, the second constituent is polyethylene glycol.

In accordance with certain embodiments of the present disclosure, the connector includes a greatest width adjacent a distal end of the multi-lumen chamber, wherein the connector can be Y-shaped and the central lumen of the connector can include a proximal end terminating within the greatest width of the connector.

In accordance with certain embodiments of the present disclosure, at least one of the first port and the second port includes an air-permeable fluid-impermeable membrane.

In accordance with certain embodiments of the present disclosure, a first tube is included and configured to be in fluid communication with the first port and the central lumen of the connector in the second state. A second tube is included and configured to be in fluid communication with the second port and the central lumen of the connector in the second state.

In accordance with certain embodiments of the present disclosure, the first and second tube form a Y-shape in the second state.

In accordance with certain embodiments of the present disclosure, a method is disclosed for producing a mixture with a mixing system to deliver to a treatment site. The mixing system can include a multi-lumen chamber including a first lumen aligned and adjacent a second lumen. The first lumen is configured to include a first constituent in a proximal portion of the first lumen and a second constituent in a distal portion of the first lumen. A first plunger can be internally positioned within the first lumen to distally move the first constituent into the distal portion to mix with the second constituent in a first state to form a first mixture. The first lumen can terminate in a first port. The second lumen can be configured to include a third constituent. A second plunger rod can be internally positioned within the second to distally move the third constituent. The second lumen can terminate in a second port. The method can include distally moving the first constituent, by the first plunger rod, to open a barrier within the first lumen thereby injecting the first constituent into the distal portion to mix with the second constituent in a first state to form a first mixture; purging air from the distal portion and a second lumen of the multi-lumen chamber through a connector port of a connector positioned on a distal end of the multi-lumen chamber, the connector including a central lumen attached to a distal end of a needle hub; and distally moving the second plunger rod causing the first mixture and the third constituent to be delivered through the first and second ports, mixed together within the central lumen of the connector to form the mixture.

In accordance with certain embodiments of the present disclosure, the second plunger rod distally moves the third constituent and the first mixture in the second state.

In accordance with certain embodiments of the present disclosure, the step of distally moving the second plunger rod to form the mixture is performed while the step of purging air through the connector port.

In accordance with certain embodiments of the present disclosure, the method includes injecting the mixture, from the connector and the needle, between a first layer of a prostate and a second layer of tissue of a rectum, wherein the mixture at least partially separates the first and second layers.

In accordance with certain embodiments of the present disclosure, at least one of the first and second lumens include a floating plunger. The method can include positioning a rib within at least the first lumen a distal end of the first lumen; and advancing distally the first plunger rod so that the first floating plunger advances distally and contacts the rib thereby developing a moment on a head of the first floating plunger causing the first floating plunger to tilt and break a fluid seal so fluid within the first lumen advances distal of the first floating plunger.

In accordance with certain embodiments of the present disclosure, the method can include preventing, by the connector port, air bubbles from entering the mixture.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIGS. 1A-1B depict the prostate, rectum, and Denonvilliers' space between the prostate and rectum.

FIG. 2 shows an exploded view of an exemplary mixing system in accordance with certain aspects of the present disclosure.

FIG. 3 shows a side, cross-section view of an exemplary mixing system in accordance with certain aspects of the present disclosure.

FIG. 4 shows an exploded view of an exemplary mixing system in accordance with certain aspects of the present disclosure.

FIG. 5 depicts a plunger assembly in accordance with certain aspects of the present disclosure.

FIG. 6A depicts a close-up perspective view of a needle hub in accordance with certain aspects of the present disclosure.

FIG. 6B depicts a close-up perspective view of an adaptor used with the needle hub of FIG. 6A in accordance with certain aspects of the present disclosure.

FIGS. 7A-7C depict example steps in a method of distally advancing a plunger rod of an example mixing system, in accordance with certain aspects of the present disclosure.

FIG. 8 depicts a partial close-up view a toggle plunger in accordance with certain aspects of the present disclosure.

FIGS. 9A-9B depict example steps in a method using an example mixing system, in accordance with certain aspects of the present disclosure.

FIGS. 10A-10D depict example steps in a method of priming an example connector, in accordance with certain aspects of the present disclosure.

FIGS. 11A-11B depict example steps in a method using an example mixing system and a primed connector, in accordance with certain aspects of the present disclosure.

FIG. 12 depicts an exemplary mixing system in accordance with certain aspects of the present disclosure.

FIGS. 13A-13B depicts a side cross-section view of the system of FIG. 12, in accordance with certain aspects of the present disclosure.

FIG. 14 depicts a flow diagram of a method of using a mixing system according to certain aspects of this disclosure.

### DETAILED DESCRIPTION

Particular aspects of the present disclosure are described in greater detail below. The terms and definitions provided herein control, if in conflict with terms and/or definitions incorporated by reference.

Particular aspects of the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

As used herein, “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%).

As used herein, “operator” can include a doctor, surgeon, or any other individual or delivery instrumentation associated with delivery or use of a mixing system as such systems are described throughout this disclosure.

The compositions herein may be used in various medical procedures, including but not limited to injected to create additional space between the rectum and prostate during treatment, for example in the Denonvilliers' space, thereby reducing rectal radiation dose and associated side effects. Certain embodiments of the disclosure include placing a filler between the radiation target tissue and other tissues. The filler can be a gel composition that increases the distance between the target tissue and other tissues so that the other tissues receive less radiation.

It is understood that “Denonvilliers' space” is a region located between the rectum and prostate. Certain embodiments provide a method of displacing a tissue to protect the tissue against the effects of a treatment involving radiation or cryotherapy. One embodiment involves using a filler mixed by a mixing system of this disclosure to displace the tissue relative to a tissue that is to receive the treatment. Another embodiment involves introducing a filler mixed by a mixing system of this disclosure to displace a first tissue and radiating a second tissue, particularly a second tissue that is close to the first tissue. In another embodiment, the method includes the steps of injecting a filler into a space between tissues; and may further include irradiating one of the tissues so that the other tissue receives less radiation than it would have in the absence of the filler.

Certain embodiments also provide methods for treating a tissue of a body by radiation. In one embodiment, the method includes the steps of injecting an effective amount of a filler into a space between a first tissue (e.g., prostate) of a body and a second tissue (e.g., rectum), which can be a critically sensitive organ; and treating the first tissue by radiation whereby the filler within the space reduces passage of radiation into the second tissue. Tissue is a broad term that encompasses a portion of a body: for example, a group of cells, a group of cells and interstitial matter, an organ, a portion of an organ, or an anatomical portion of a body, e.g., a rectum, ovary, prostate, nerve, cartilage, bone, brain, or portion thereof.

The gel of the filler can include polymeric materials which are capable of forming a hydrogel may be utilized. In one embodiment, the polymer forms a hydrogel within the body. A hydrogel is defined as a substance formed when an organic polymer (natural or synthetic) is cross-linked via covalent, ionic, or hydrogen bonds to create a three-dimensional open-lattice structure which entraps water molecules to a gel. Naturally occurring and synthetic hydrogel forming polymers, polymer mixtures, and copolymers may be utilized as hydrogel precursors.

In some aspects, the hydrogel can be formed by a composition formed by mixing constituents together (E.g., accelerant fluid, diluent, and PEG together) and may comprise one or more polysaccharide compounds or a salt thereof. For example, the composition may include a cellulose compound such as carboxymethyl cellulose (CMC) or salt thereof (e.g., CMC) sodium, xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be cross-linked with a suitable crosslinking compound, such as butanediol diglycidyl ether (BDDE). In some aspects, the polysaccharide may be a homopolysaccharide or a heteropolysaccharide

The present disclosure also provides mixing systems to form the gel composition and corresponding medical devices for use and/or delivery to a treatment site of a patient. According to some aspects of the present disclosure, the mixing system may include a plurality of reservoirs with respective lumens. Collectively, the lumens therein may serve as a container for constituents to mix the gel composition of this disclosure. Suitable reservoirs may include, for example, syringes (e.g., a syringe barrel compatible with a manual or automatic injection system) and other fluid containers configured for use with a suitable injection needle. Exemplary materials suitable for the reservoir include, but are not limited to, cyclic olefin polymer, polypropylene, polycarbonate, polyvinyl chloride, and glass. In some aspects, one of these materials (e.g., cyclic olefin copolymer specifically) can have a coating applied to it, such as SiO₂), which is advantageous so the coating can perform as a primary oxygen barrier, behave as a glass-like layer, and can be applied using a vapor deposition process.

According to some aspects of the present disclosure, the compositions may include at least one accelerant (e.g., an activating agent) combined with a precursor mixed from a diluent (e.g., mostly water) and polyethylene glycol (PEG). In some examples, the composition may be or include a gel with a desired gel strength and/or viscosity, such as a biocompatible gel suitable for injection (e.g., through a needle).

The hydrophilic polymer can be any gelling agent(s), including natural ones or synthetic in origin, and may be anionic, cationic, or neutral. Non-limiting examples of the gelling agents include polysaccharides such as gellan gum, xanthan gum, gum arabic, guar gum, locust bean gum, alginate, and carrageenans.

The concentrations of gelling agent(s) in the composition described in this disclosure may range from about 0.01% to about 2.0% by weight with respect to the total weight of the composition, such as from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, the composition may have a viscosity ranging from about 0.001 Pa·scal-second (Pa·s) to about 0.100 Pa·s at a shear rate of 130 s⁻¹, such as, e.g., from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, the composition may have a viscosity ranging from about 0.001 Pa·s to about 0.050 Pa·s at a shear rate of 768 s⁻¹, such as, e.g., from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s^{'11}. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The mixing system herein may include or be removably connected to one or more needles. In some examples, the needle may be a hypodermic needle, and may range from a size of 7 gauge (4.57 mm outer diameter (OD), 3.81 mm inner diameter (ID)) to 33-gauge (0.18 mm OD, 0.08 mm ID), e.g., a size of 16 gauge (1.65 mm OD, 1.19 mm ID), 18 gauge, 21 gauge (0.82 mm OD, 0.51 mm ID), 22 gauge (0.72 mm OD, 0.41 mm ID), 23 gauge (0.64 mm OD, 0.33 ID), or 24 gauge (0.57 mm OD, 0.31 mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

According to some aspects of the present disclosure, the filler compositions herein, e.g., the compositions prepared by the methods herein may have sufficient strength, e.g., gel strength, to withstand the forces and thus minimizing the effects of the forces on the continuity of the three-dimensional gel network. In the meantime, the composition with sufficient strength may have a viscosity suitable for injection, e.g., a viscosity that does not render the composition stuck in the reservoir(s), delivery lumen, or a needle connected therewith.

According to some aspects of the present disclosure, the composition may maintain its three-dimensional structure until the gel is injected through a needle, whereupon the structure may form fragments of the original continuous, three-dimensional network. Those gel fragments may have a diameter corresponding to the diameter of the injection needle, such that the fragments are as large as possible in-vivo to retain as much of the three-dimensional structure of the gel as possible. Injection of these larger-sized particles or fragments is believed to increase the amount of time the gel remains within the tissue.

The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lbf to about 25 lbf, such as from about 10 lbf to about 20 lbf, e.g., about 15 lbf. The loads measured for a given gel concentration may vary for different needles and flow rates.

According to some aspects of the present disclosure, the size of the needle may be chosen based on the viscosity and/or components of the composition, or vice versa. According to some aspects of the present disclosure, the size of the needle may be 23 gauge or 25 gauge. In some cases, a larger size of 18-gauge, 20 gauge, 21 gauge, or 22 gauge may be used to inject the compositions herein.

According to some aspects of the present disclosure, the mixing system of this disclosure can be included in a kit for introducing a filler into a patient, whereby the filler can include any of the gel compositions of this disclosure. Kits or systems for mixing a gel composition of this disclosure, such as hydrogels, may be prepared so that the precursor(s) and any related activating agent(s) are stored in the kit with diluents as may be needed. Applicators may be used in combination with the same. The kits can be manufactured using medically acceptable conditions and contain components that have sterility, purity and preparation that is pharmaceutically acceptable. Solvents/solutions may be provided in the kit or separately. The kit may include syringes and/or needles for mixing and/or delivery. The kit or system may comprise components set forth herein.

During some examples of use, once saline has been injected to the treatment site, a mixing system can be connected to a needle (e.g., an 18-gauge spinal needle) to then inject a 5-10 mm layer of filler (e.g., gel composition) along the posterior wall of the prostate between the prostate and rectum. Once the filler has been injected into the space between the rectum and prostate, ultrasound images can be obtained.

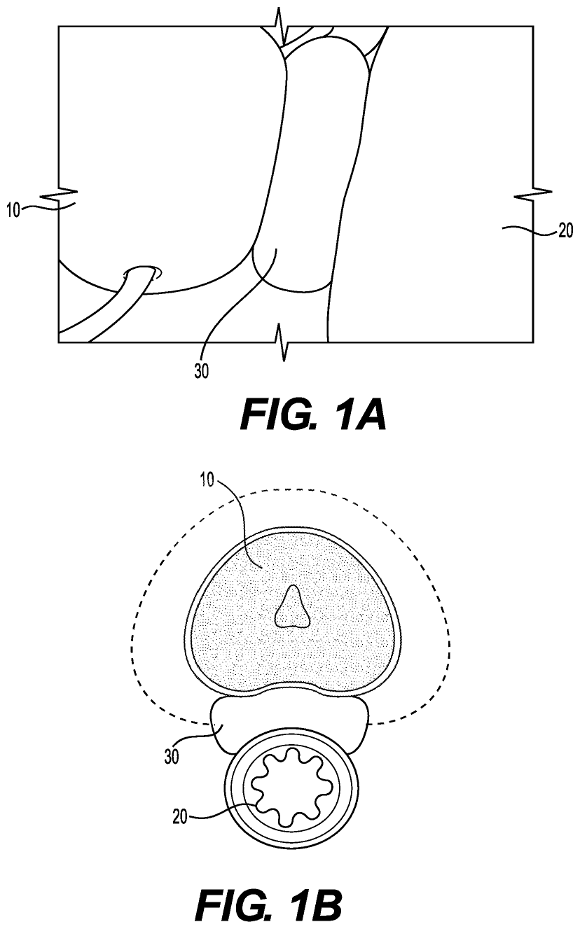

Turning to the drawings, FIG. 1A is a perspective view and FIG. 1B is a partial cross-section view illustrating example filler 30, in the form of a gel composition having been delivered by the mixing system of this disclosure between rectum 20 and prostate 10 of a patient in Denonvilliers' space.

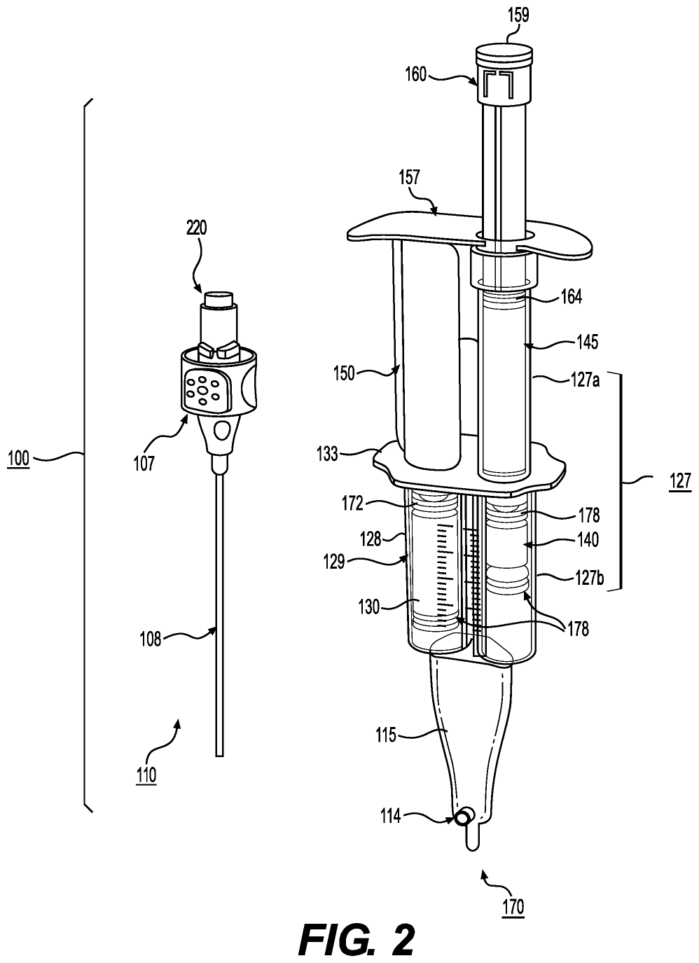

FIG. 2 shows an exploded view of an exemplary mixing system 100 in accordance with certain aspects of the present disclosure for mixing a gel composition. The system 100 can include a needle assembly 110 attachable to the main assembly 170 of system 100. Needle assembly 110 can include needle 108, which can be any needle of this disclosure suitable for hydrodissection as well as delivering filler 30 (e.g., the gel composition) to the treatment site.

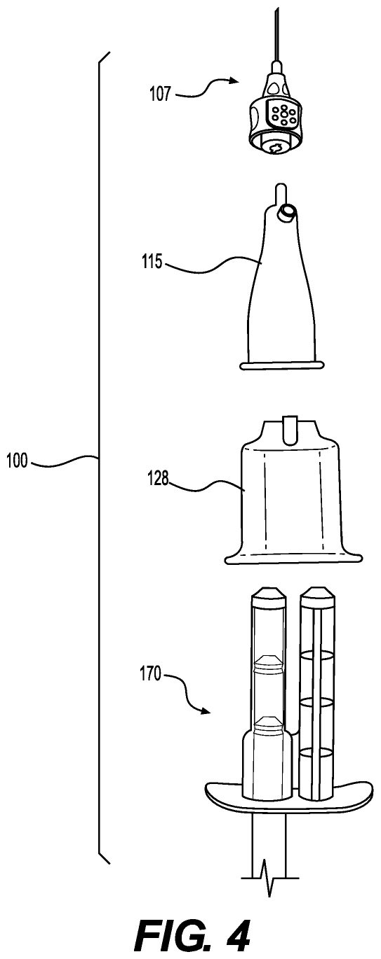

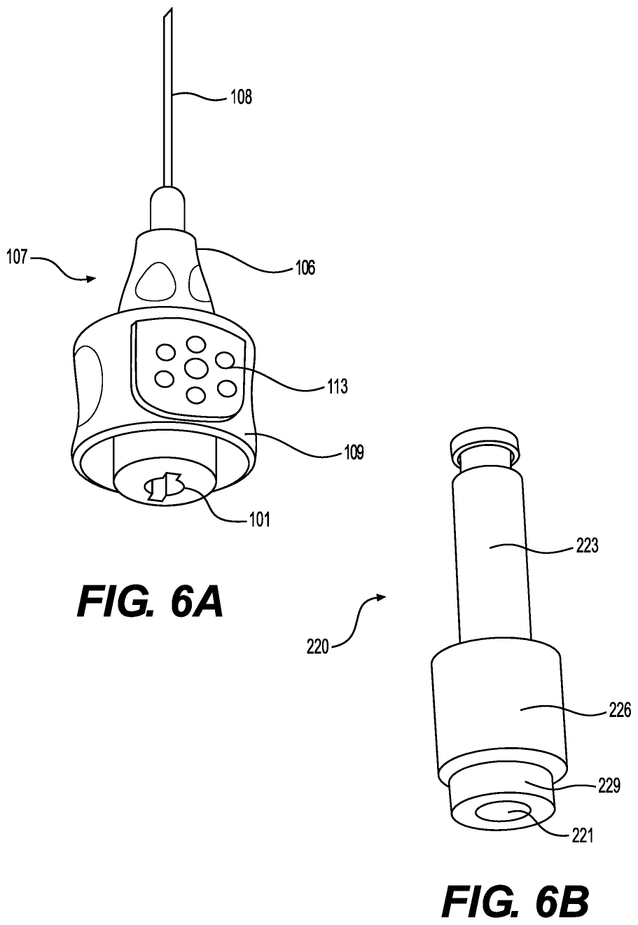

A proximal end of needle 108 can be connected to a distal end of a needle hub 107 (e.g., needle 108 can be overmolded to connect to hub 107), which can be attached to a distal end of connector 115. Needle hub 107 is shown more clearly in FIGS. 4 and 6A, where it includes a lower housing 109 configured to be gripped and squeezed by user U. An externally positioned button 113 can be positioned on an outer surface of lower housing 109. Button 113 can be configured so that an actuating squeeze or other movement by user causes latches of hub 107 to attach or release from a distal end of connector 115 to open and close corresponding connecting latches of connector hub 107 with respect to its engagement with connector 115. However, other coupling approaches between hub 107 and connector 115 are contemplated as needed or required. For example and without limitation, snap fit connectors, magnetic connectors, female-male connectors, hook and loop fasteners and the like are contemplated.

Hub 107 may also include a transitional portion 106 through which needle 108 can be inserted. Portion 106 can include a diameter smaller than housing 109. In some examples, portion 106 can be tapered and/or include a textured outer surface. A central tubular lumen 101 can pass through hub 107 and be in fluid communication with needle 108, when needle 108 is connected to hub 107. Lumen 101 can include a luer fitting configured to receive a distal end of syringe 200 and a distal end of system 100.

In certain aspects, hub 107 can be configured to attach to syringe 200 via adaptor 220. Adaptor 220 is shown in FIG. 2 and more closely in FIG. 6B with portion 229 configured to sit within a distal end of syringe 200. Adaptor 220 can include a distal portion 223 configured to removably insert into and attach with hub 107. Central portion 226 is positioned between portions 229 and 223 and includes an outer diameter greater than portions 223 and 229. In this respect, a lip or ridge is created between portions 223 and 226 that acts as a stop to prevent housing 109 from sliding therepast when hub 107 is attached with adaptor 226. Similarly, a lip or ridge is created between portions 226 and 229 that acts as a stop to prevent a distal end of syringe 200 from sliding therepast when adaptor 220 is attached to syringe 200.

Turning back to FIG. 2, main assembly 170 of system 100 can include a multi-lumen chamber formed by a first lumen 127 inside a first barrel and a second lumen 129 inside a second barrel. Each lumen 127, 129 can be oriented parallel with the other, running side-by-side. A first plunger stopper 164 can be located at a distal end of a first plunger rod 160. Rod 160 can be advanceable within lumen 127. In some aspects, rod 160 can be advanced by button 159 positioned on a proximal end of rod 160.

Lumen 127 can be divided into a proximal portion 127a and a distal portion 127b by a second plunger stopper 178 can be positioned within lumen 127, including separate portions 127a, 127b. Stopper 178 can be a floating plunger. Portions 127a and 127b can each include one or more constituents (e.g., a fluid, liquid, powder, or some combination thereof). Distally moving rod 160 can cause stopper 164 to advance constituent(s) of portion 127a so as to open a barrier associated with stopper 178 thereby allowing constituents of each portion 127a, 127b to intermix and form precursor. As used herein, the term “fluid” as it relates to constituents of system 100 is defined broadly and can include liquids, gels and particulate matter such as granules, pellets, or powders, or any combination of liquids, gels, oils, and/or particulate matter (e.g., granules, pellets, or powders). In some example, constituent(s) of portion 127a can be a diluent fluid solution (e.g., constituent 145) and portion 127b can include an activating agent (e.g., constituent 140), such as PEG or any other agent mixable with diluent to form precursor. The diluent can be a branched polymer having a plurality of succinimidyl termini dissolved in a low pH (4.0) containing a low molecular weight precursor comprising nucleophiles, though other diluent fluid solutions are contemplated within the scope of this disclosure. Once mixed together, precursor solution 145′ can be formed in portion 127b.

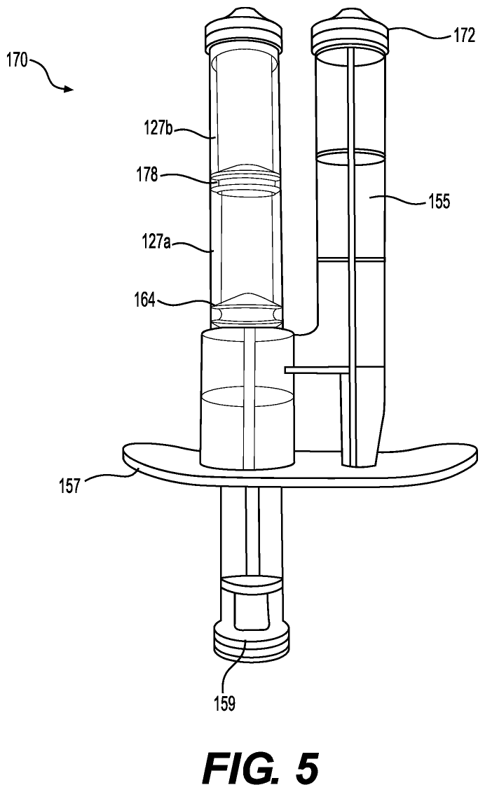

Lumen 129 can similarly include a plunger rod 155 slidable therein. A distal end of rod 155 can include a stopper 172. A proximal end of rod 155 can include an actuating flange 157 configured so that a user can a press thereon to drive rod 155 proximally or distally. As seen clearly in FIG. 5, plunger rod 155, flange 157, rod 160, and portions 127a, 127b can be partially or entirely integrally formed together to form plunger assembly 170. Assembly 170 can be assembled to form lumen 129 by positioning distal ends of rods 155, 160 with distal ends of receiver 128, as shown more clearly in FIGS. 4 and 5. Receiver 128 can include an open upper end with a flange 133 and plurality of smaller openings opposite thereof and configured to receive ports 138 of assembly 170. In FIG. 4 specifically, plunger assembly 170 is shown proximal and just prior to being assembled through the open upper end of receiver 128. It is also contemplated that additional floating plunger stoppers 178 can be include in system 100, including a stopper 178 distal of stopper 172 in lumen 129. Stopper 178 can be a floating plunger capable of being toggled when activated by pressure so as to allow the fluid of respective lumen 127, 129 to pass thereby.

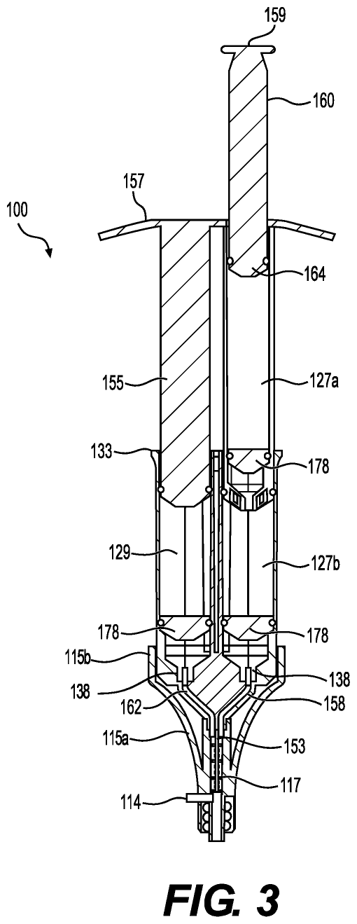

Turning back to FIG. 3, ports 138 of lumen 127 and lumen 129 can be configured to permit egress of fluids from respective lumens 127, 129. Also optionally, in a first state before mixing, the system 100 can include a retainer 150 removably positioned between flanges 133 and 157 so as to prevent unwanted movement rod 155. In some aspects, once assembly 170 is nested within receiver 128, lumen 129 is formed between an outer surface of portion 127 and an inner surface of receiver 128. Constituent 130 (e.g., accelerant fluid) can be positioned therein, as shown clearly in FIG. 2 and FIGS. 9A-10A, and stopper 172 of rod 155 can advance constituent 130 to mix with precursor 145′ once distal of ports 138.

In some aspects, while flange 157 is permanently or temporarily attached to button 159 of rod 160, distally advancing flange 157 can distally advance both stopper 172 as well as rod 160, stopper 164, and/or stopper 178 so that the precursor 145′ and constituent 130 are capable of egressing through respective ports 138 and mixing together distal thereof in a static mixer. In some aspects, flange 157 can include an opening sized to permit rod 160 to slide therethrough. However, button 159 can be larger than the opening so as to prevent button 159 from sliding distal of flange 157 and ensure that once button 159 and flange 157 are aligned or otherwise attached, flange 157 being distally advanced can drive both rod 160 and rod 155 simultaneously. In some aspects, flange 157 and button 159 can be secured together when button 159 is moved to a distalmost position (e.g., via L-shaped elements of button 159).

Referring now to connector 115, as shown in FIGS. 2-4, it can be seen that connector 115 includes a distal portion 115a and a proximal portion 115b. Portion 115b can be integrally formed with (e.g., continuous with, injection molded, etc.) or insertable into an open upper end of portion 115a to nest therewith and form connector 115. Portion 115a can be substantially hollow with a tapered or Y-shape profile for its outer surface. Portion 115a can terminate in a distal end with a central lumen 117.

As seen more clearly in FIG. 3, each of lumens 127, 129 can be in fluid communication with a proximal end of lumen 117 of connector 115. Portion 115b can receive port 138 of each lumen 127, 129 and provide a fluid path for each to a proximal end of lumen 117. Lumen 117 can include a static mixer 153 so that fluid from respective lumens 127, 129 can mix together and form the gel composition to be delivered through needle 108.

In some aspects, portion 115b can include a tube 158 (e.g., a hypotube) with a proximal end configured in fluid communication with lumen 127 and pierce a corresponding membrane or seal of port 138. Portion 115b can also include tube 162 (e.g., a hypotube) with a proximal end configured in fluid communication with lumen 129 and pierce a corresponding membrane or seal 136 of port 138. Vent 114 can be included in connector 115 to facilitate purging any unwanted air stored in the fluid path of connector 115 or lumens 127, 129. In some aspects, vent 114 can be distal of mixer 153 and formed integrally with an outer surface of connector 115. In this respect, once precursor 145′ is in position in lumen 127 and constituent 130 is positioned in lumen 129 distally moving rod 155 can cause precursor 145′ and constituent 130 to egress through respective ports 138 and respective tubes 158, 162 to mix with each other in lumen 117. In some aspects, air can be purged from lumen 127, lumen 129, and/or connector 115 through vent 114 during mixture of precursor 145′ and constituent 130 in lumen 117. Tubes 158, 162 can form a Y-shape, as in FIG. 3, though any other shape can be used as needed or required.

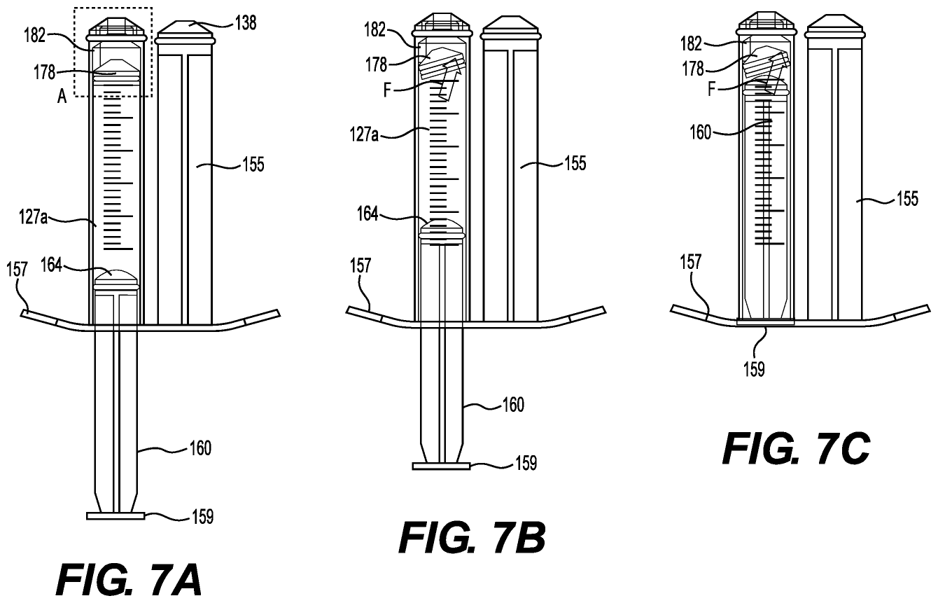

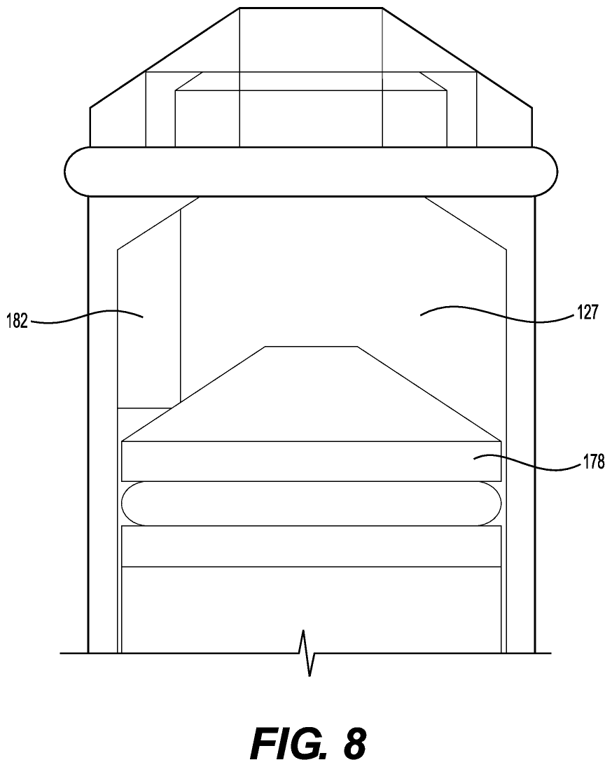

In some aspects, at least one of lumens 127, 129 can include an internally positioned rib that can facilitate toggling of plunger 178. For example, in FIGS. 7A-7C and 8, side cross-sectional views are provided depicting example rib 182 causing plunger 178 to toggle. Rib 182 can be internally positioned in a distal end of portion 127a. Rib 182 can be build-up material or a feature detachable from lumen 127. In FIG. 7A, rib 182 is shown in contact with plunger 178 while stopper 164 of rod 160 is adjacent flange 157 in a fully cocked position ready for distal advancement. Between FIGS. 7B-7C, rod 160 advances stopper 164 causing internal pressure of lumen 127 to increase and toggle plunger 178. In some examples, as a result of increased pressure, a distal end of plunger 178 can be tapered so that as its leading edge contacts rib 182, plunger 178 develops a moment. This toggling action can cause a previous seal between plunger 178 and lumen 127 to break so fluid in portion 127a can move distal thereof (e.g., through a fluid port) and into portion 127b, as denoted by the fluid path F.

FIG. 8 shows a partial cross-section, close-up view of section A of FIG. 7A depicting an example rib 182. However, rib 182 and/or plunger 178 can come in any number of shapes and sizes so as to induce a moment and related toggle action between rib 182 and plunger 178. As shown, a tip of plunger 178 can be pointed or tapered so that contacting rib 182 causes the previously-described moment and toggle. However, rib 182 could be tapered or otherwise include a contoured lower surface configured to induce any shaped tip of plunger 178 toggle, as needed or required.

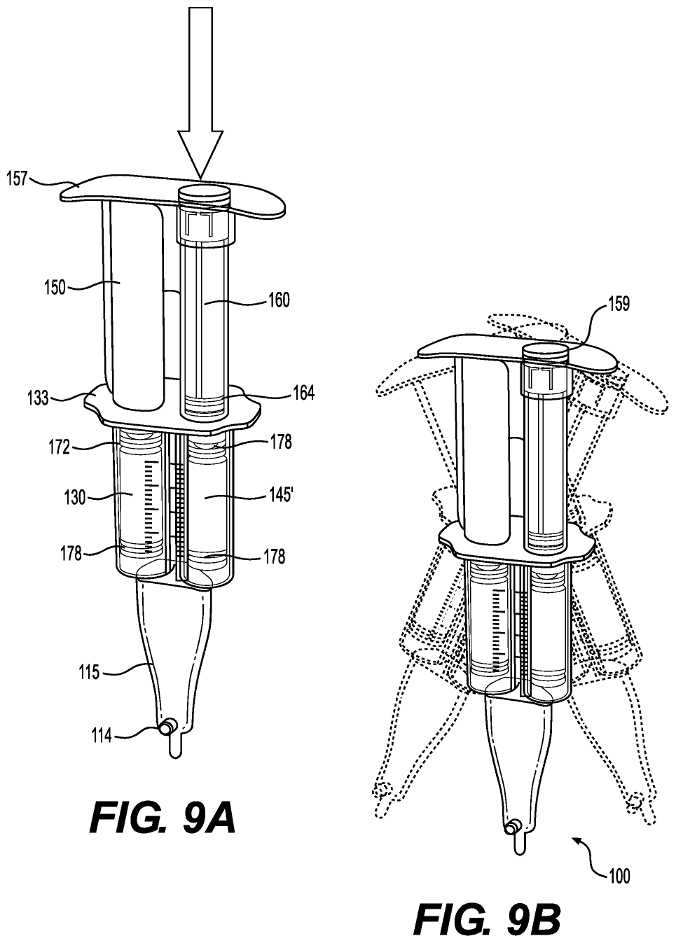

Now, turning to FIGS. 9A-11B are example steps of a process of using system 100 according to certain aspects of this disclosure. While certain steps are shown as a sequence between each figure, in other embodiments, fewer steps are contemplated and the order by which steps are performed can be different than what is illustrated. In FIG. 9A, system 100 is introduced in a first state with rod 155 fully retracted and retainer 150 positioned between flanges 133, 157. In the depicted configuration, rod 155 is incapable of distally moving as a result of retainer 150 being wedged between flanges 133, 157. In contrast, rod 160 has been advanced distally as denoted by the downward arrow so that constituent 145 has been moved by stopper 164 and barrier of stopper 178 moved causing constituent 145 to mix with constituent 140.

In FIG. 9B, aspects of system 100 can be shaken back and forth to ensure precursor 145′ forms as a result of mixing between constituent 145 and constituent 140, while constituent 130 remains in lumen 129. Preferably, the shaking action of FIG. 9B is done while the ports 138 are oriented generally upward. However, the shaking to effect proper mixing of precursor 145′ can be performed in other orientations (e.g., generally downward, etc.), as needed or required.

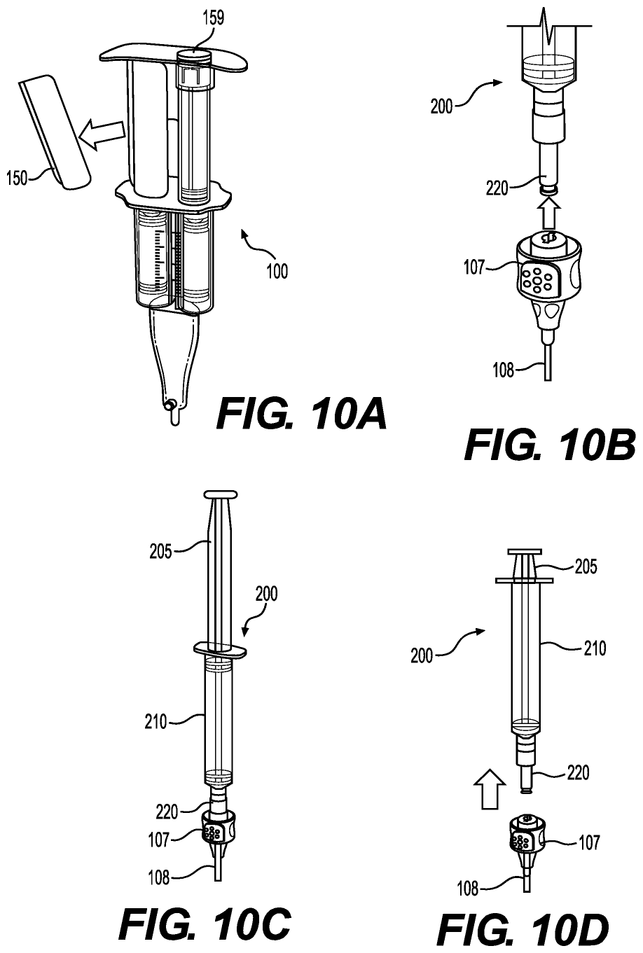

In FIG. 10A, with precursor 145′ formed and constituent 130 in lumen 129, retainer 150 can be removed and flange 157 can be distally advanced now. Separately, in FIG. 10B, hub 107 is shown being connected to syringe 200 via adaptor 220. While not shown, during use hub 107 is contemplated to be connected to needle 108 for hydrodissection at the treatment site with saline from the syringe 200. After hydrodissection with syringe 200 (FIG. 10C), needle 108, adaptor 220, and hub 107 can be released, as shown in FIG. 10D. In some examples, button 113 of hub 107 can be used to attach and detach hub 107 from adaptor 220 and syringe 200.

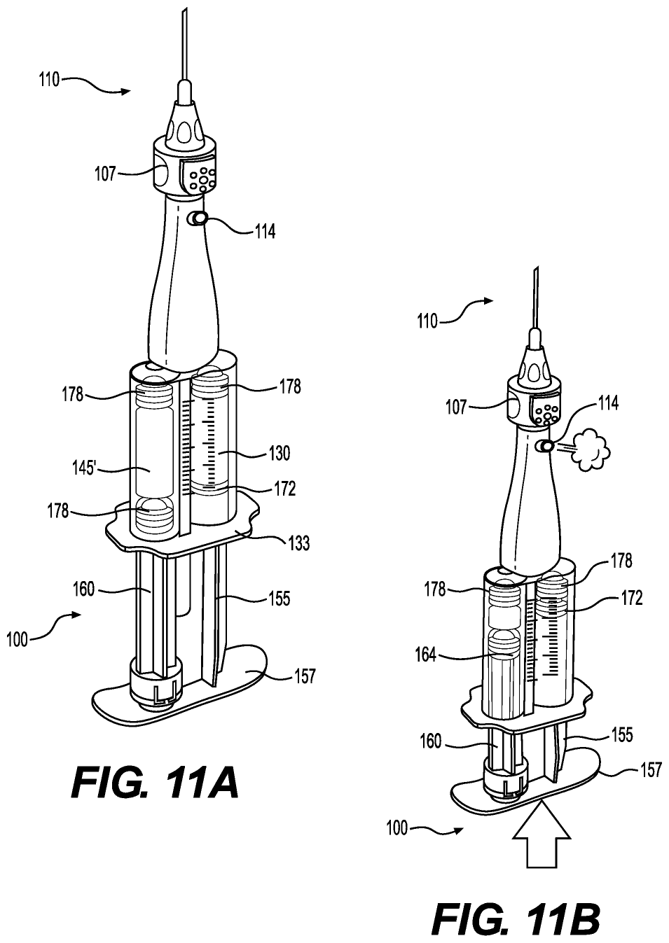

Turning to FIG. 11A, aspects of system 100 are now connected to needle assembly 110 via hub 107 and connector 115. It is understood that connection between hub 107 and 115 occurs while needle 108 and hub 107 are in position at the treatment site. With precursors 145′ and constituent 130 in position in respective lumens and retainer 150 removed, a user U can advance flange 157 distally so that corresponding rods 155, 160 distally advance respective stoppers 164,172. In some examples, pressure within lumen 127 can be increased by advancing rods 155 and/or 160 so as to toggle any corresponding plunger(s) 178 and advance precursor 145′ from lumen 127 and constituent 130 from lumen 129, through ports 138, and into the central lumen 117 of connector 115. As long as flange 157 continues advancing, as in FIG. 11B, precursor 145′ and constituent 130 can mix within central lumen 117 and continue egressing through needle 108 and ultimately to the treatment site.

Advantageously, vent 114 can purge any unwanted air from connector 115 from the distal ends of ports 138 or lumen 117, as shown in FIG. 11B, thereby preventing air bubbles from entering the patient at the treatment site with the gel composition. Optionally, lumen 117 can include a static mixer 135 configured to thoroughly mix the fluids together to form the gel composition to be delivered to the treatment site. Aspects of system 100 as shown being used in FIGS. 9A-11B are relatively easy to assemble and minimizes potential unintentional gel mixing errors prior to delivery.

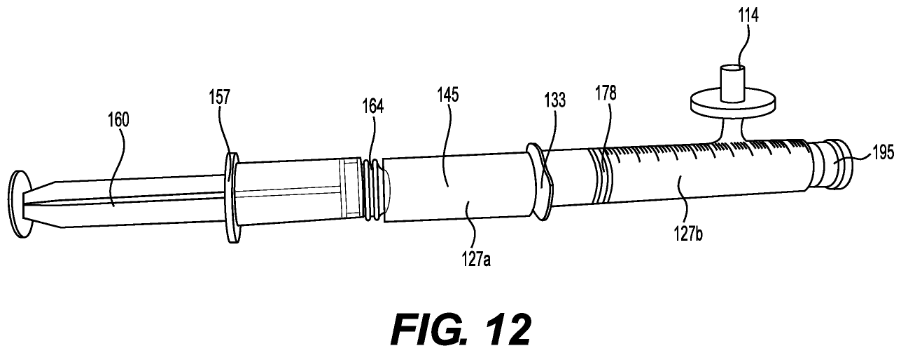

FIG. 12 illustrates a side-perspective view of an example embodiment of lumen 127 including vent 114, as opposed to vent 114 being located on connector 115, as previously shown. Vent 114 in FIG. 12 can be configured to purge any unwanted air from corresponding lumen 127 to prevent air bubbles from entering the patient at the treatment site with the gel composition. Optionally, cap 195 can be positioned on a distal end of system 100. While not shown, it is contemplated that lumen 129 could also include a vent of its own or be in fluid communication with vent 114 of FIG. 12. Other vents of this example could also be included for purging unwanted air, including a vent similar to vent 114 previously shown with connector 115.

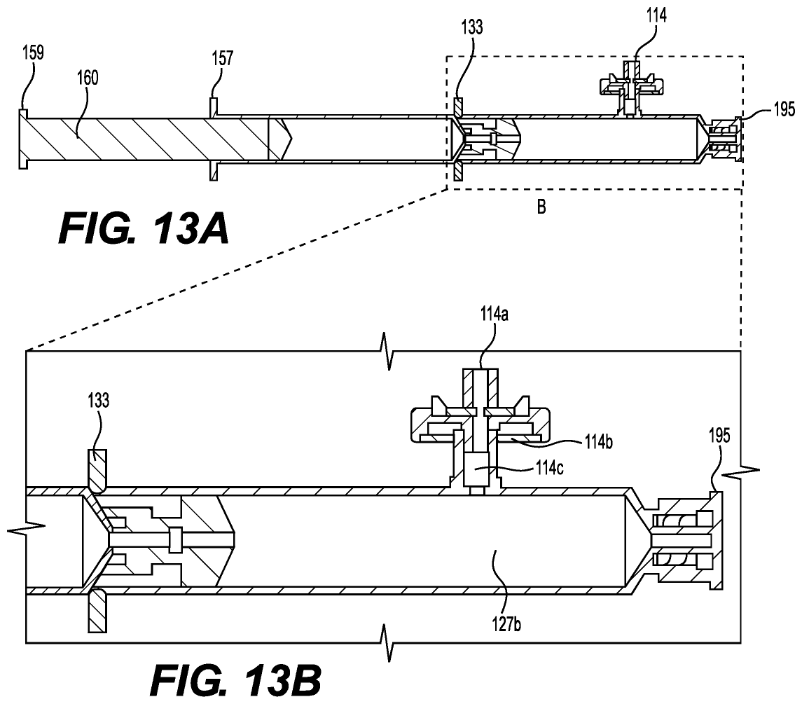

FIGS. 13A-13B depicts a side cross-section view of the system of FIG. 12, in accordance with certain aspects of the present disclosure. In particular, FIG. 13A shows example system 100 with the vent 114 of FIG. 12 and FIG. 13B shows a close-up of section B. Vent 114, as shown in FIG. 13B, can include an entry port 114a, a membrane 114b, and distal exhaust port 114c. Port 114a in fluid communication with lumen 127. In some embodiments, port 114a can extend orthogonally from lumen 127 and be positioned proximal of port 138. Membrane 114b can be distal of port 114a and proximal of port 114c, whereby membrane 114b can be a one-way valve or seal openable to release air when predetermined pressure is achieved. Membrane 114b can be constructed from a polymer, such as Polytetrafluoroethylene (PTFE). In some examples, membrane 114b can be opened manually be user U wishing to purge air from lumen 127. In some examples, membrane 114b can include a diameter larger than ports 114a, 114c and form a T-like shape. Membrane 114b can be oriented so that air purged therefrom is purged out of membrane and back towards the outer surface of lumen 127. However, the example of FIGS. 13A and 13B are merely for illustrative purposes, and other orientations and shapes of membrane 114b are contemplated as needed or required, within the scope of this disclosure.

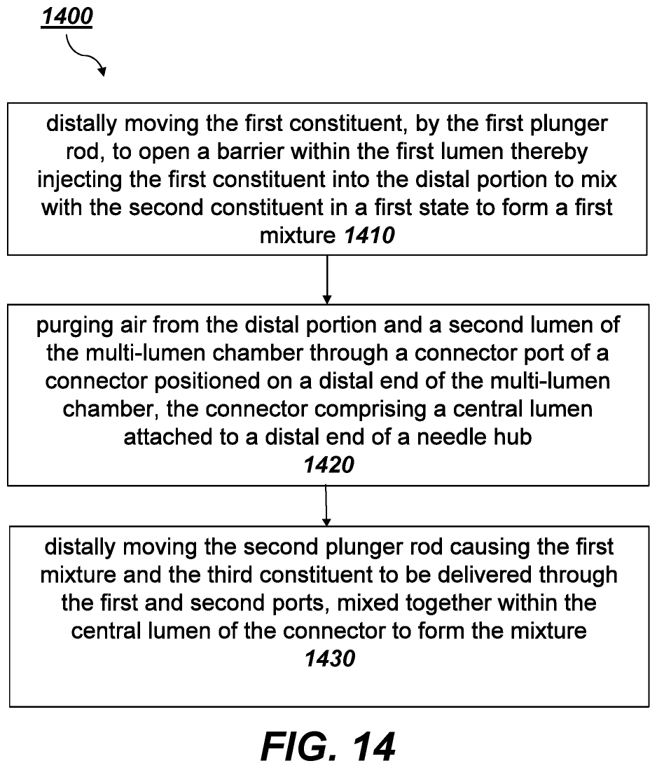

FIG. 14 depicts a method or use 1400 of any of the herein disclosed mixing systems. Step 1410 of method 1400 can include distally moving the first constituent, by the first plunger rod, to open a barrier within the first lumen thereby injecting the first constituent into the distal portion to mix with the second constituent in a first state to form a first mixture. Step 1420 of method 1400 can include purging air from the distal portion and a second lumen of the multi-lumen chamber through a connector port of a connector positioned on a distal end of the multi-lumen chamber, the connector comprising a central lumen attached to a distal end of a needle hub. Step 1430 of method 1400 can include distally moving the second plunger rod causing the first mixture and the third constituent to be delivered through the first and second ports, mixed together within the central lumen of the connector to form the mixture. Method 1400 can end after step 1430. In other embodiments, additional steps according to the examples described above can be performed.

Other aspects and embodiments of the present disclosure will be apparent to those skilled in the art from consideration of the specification and practice of the embodiments disclosed herein. While certain features of the present disclosure are discussed within the context of exemplary procedures, the compositions, systems, and methods may be used for other medical procedures according to the general principles disclosed. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein. It is intended that the specification and examples be considered as exemplary only, with a true scope and spirit of the present disclosure being indicated by the following claims.

What is claimed is:

1. A system for producing a mixture to deliver to a treatment site, comprising:
a connector comprising a central lumen attachable to a proximal end of a delivery system, the connector comprising a connector vent in fluid communication with the central lumen configured so that air in the connector is expellable therethrough;
a multi-lumen chamber connected to a proximal end of the connector and comprising a first lumen aligned and adjacent a second lumen;
the first lumen configured to comprise a first constituent in a proximal portion of the first lumen and a second constituent in a distal portion of the first lumen, a first plunger internally positioned within the first lumen to distally move the first constituent into the distal portion to mix with the second constituent in a first state to form a first mixture, the first lumen terminating in a first port;
the second lumen configured to comprise a third constituent, a second plunger rod internally positioned within the second to distally move the third constituent, the second lumen terminating in a second port;
wherein distally moving the second plunger rod causes the third constituent to be delivered through the first and second ports, vent air from system through the connector vent, mix within the central lumen of the connector to form the mixture, and delivered through the delivery system.

2. The system of claim 1, wherein the second plunger rod distally moves the third constituent and the first mixture in the second state.

3. The system of claim 1, wherein distally moving the second plunger rod causes the first mixture and the third constituent to be delivered through the first and second ports while venting air from the system through the connector vent.

4. The system of claim 1, wherein at least one of the first and second lumens comprise a floating plunger, wherein at least one of the first and second floating plungers are toggleable when a predetermined pressure is achieved within a respective lumen.

5. The system of claim 1, the delivery system comprising a needle assembly removably connected to the connector, the needle assembly comprising the needle and a needle hub positioned at a proximal end of the needle.

6. The system of claim 5, wherein at least one of the first and second lumens comprise a floating plunger,
wherein the first lumen comprises an internally positioned rib at or adjacent a distal end of the first lumen,
wherein as the first plunger rod is advanced distally, the first floating plunger advances distally and contacts the rib to develop a moment on a head of the first floating plunger causing the first floating plunger to tilt and break a fluid seal so fluid within the first lumen can advance distal of the first floating plunger.

7. The system of claim 1, wherein the central lumen of the connector comprises a static mixer and wherein the connector vent extends orthogonally from the central lumen to outside air distal of the static mixer.

8. The system of claim 1, wherein the connector vent further comprises a one-way valve configured to permit egress of air from the central lumen.

9. The system of claim 1, wherein the first lumen comprises a vent proximal of the first port and orthogonal to the first lumen configured to permit egress of air from the first lumen.

10. The system of claim 1, wherein the first mixture and the third fluid mixed together forms a hydrogel, and wherein the connector vent is configured to prevent air bubbles from entering the hydrogel.

11. The system of claim 1, wherein the connector vent comprises an air-permeable-fluid-impermeable membrane.

12. The system of claim 1, wherein the first lumen, the first plunger assembly, and the second plunger rod are each comprised in a plunger assembly; and
wherein the plunger assembly is nestable within the second lumen.

13. The system of claim 1, wherein the second constituent is polyethylene glycol.

14. A method for producing a mixture with a mixing system to deliver to a treatment site, the mixing system comprising:
a multi-lumen chamber comprising a first lumen aligned and adjacent a second lumen;
the first lumen configured to comprise a first constituent in a proximal portion of the first lumen and a second constituent in a distal portion of the first lumen, a first plunger internally positioned within the first lumen to distally move the first constituent into the distal portion to mix with the second constituent in a first state to form a first mixture, the first lumen terminating in a first port;
the second lumen configured to comprise a third constituent, a second plunger rod internally positioned within the second to distally move the third constituent, the second lumen terminating in a second port, the method comprising:
distally moving the first constituent, by the first plunger rod, to open a barrier within the first lumen thereby injecting the first constituent into the distal portion to mix with the second constituent in a first state to form a first mixture;
purging air from the distal portion and a second lumen of the multi-lumen chamber through a connector port of a connector positioned on a distal end of the multi-lumen chamber, the connector comprising a central lumen attached to a distal end of a needle hub; and
distally moving the second plunger rod causing the first mixture and the third constituent to be delivered through the first and second ports, mixed together within the central lumen of the connector to form the mixture.

15. The method of claim 14, wherein the second plunger rod distally moves the third constituent and the first mixture in the second state.

16. The method of claim 14, wherein the step of distally moving the second plunger rod to form the mixture is performed while the step of purging air through the connector port.

17. The method of claim 14, further comprising:
injecting the mixture, from the connector and the needle, between a first layer of a prostate and a second layer of tissue of a rectum, wherein the mixture at least partially separates the first and second layers.

18. The method of claim 14, wherein at least one of the first and second lumens comprise a floating plunger, the method further comprising:
positioning a rib within at least the first lumen a distal end of the first lumen; and
advancing distally the first plunger rod so that the first floating plunger advances distally and contacts the rib thereby developing a moment on a head of the first floating plunger causing the first floating plunger to tilt and break a fluid seal so fluid within the first lumen advances distal of the first floating plunger.

19. The method of claim 14, further comprising:
preventing, by the connector port, air bubbles from entering the mixture.
